(19) United States
(12) Patent Application Publication
(10) Pub. No.: US 2008/0241250 A1
(43) Pub. Date: Oct. 2, 2008

(54) IN VIVO BIOREACTORS AND METHODS OF MAKING AND USING SAME

(76) Inventors: Pieter J. Emans, Maastrietht (NL); Prasad Venkatram Shastri, Nashville, TN (US)

Correspondence Address: Ballard Spahr Andrews & Ingersoll, LLP, SUITE 1000, 999 PEACHTREE STREET, ATLANTA, GA, 30309-3915, US

(21) Appl. No.: 11/937,068

(22) Filed: Nov. 8, 2007

Related U.S. Application Data
(60) Provisional application No. 60/864,858, filed on Nov. 8, 2006.

(51) Int. Cl. A61K 9/10; A61K 35/12; A61P 19/00

(52) U.S. Cl. 424/488; 424/484; 424/93.7

(57) ABSTRACT

The present invention relates to an in vivo method of promoting the growth of autologous cartilage and bone tissue, including tissue that can be explanted to other locations in the subject.

### CROSS-REFERENCE TO RELATED APPLICATIONS

This application claims benefit of U.S. Provisional Application No. 60/864,858, filed Nov. 8, 2006, which is hereby incorporated herein by reference in its entirety.

### BACKGROUND OF THE INVENTION

Annually, millions of individuals in North America and Northern Europe suffer from damage to articular cartilage due to lifestyle and sports related injuries, with about 25% of them requiring knee arthroplasty (Solchaga, L. A., et al, 2001). If left untreated, this injury can lead to early onset of osteoarthritis (Mollenhauer, J. A. et al, 2002).

Damage to articular cartilage is typically treated using two distinct approaches. The first one called osteoarticular transfer system (OATS), alternatively known as Mosaicplasty, involves harvesting of osteochondral plugs from the non-weight bearing regions of the condyle and inserting them in the areas of damage. Autologous osteochondral grafting represents a promising approach, but is limited by the availability of the grafts (Schaefer, D., et al, 2002), is not capable of inducing repair of the damaged area, and is limited to focal defects (<3 mm) that are not full-thickness and do not have propensity to ossify (Gross A E, 2003).

In the past decade, a new technique involving transplantation of expanded chondrocytes under a periosteal flap has gained popularity (Brittberg M, et al, 1994; Brittberg M, et al, 1996). The primary advantage of the Carticel® procedure is the ability to treat a large-sized defect (Gross A E, 2003). However, in vitro engineering of osteochondral grafts using human culture-expanded autologous cells poses several challenges, such as variability in tissue quality, cost, and complex logistics.

While these procedures provide temporary relief of the symptoms of osteoarthritis, the quality of the cartilage is typically sub par and is composed of fibro cartilage, while natural articular cartilage is Hyaline in nature. Thus, there is a need to develop new methodologies and materials that can foster the development of natural hyaline cartilage in a more expeditious and cost-effective manner.

### BRIEF SUMMARY OF THE INVENTION

In accordance with the purpose of this invention, as embodied and broadly described herein, this invention relates to an in vivo method of promoting the growth of autologous cartilage and bone tissue, including tissue that can be explanted to other locations in the subject.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1A shows representative COL2 stained tissue section from In vivo Bioreactor (IVB) filled with Hyaluronic acid (HA)-Gel+liposome. The side of the graft that was cored out for transplantation into an osteochondral defect is indicated by the letters SD. FIG. 1B shows is a higher magnification image of the box in A.

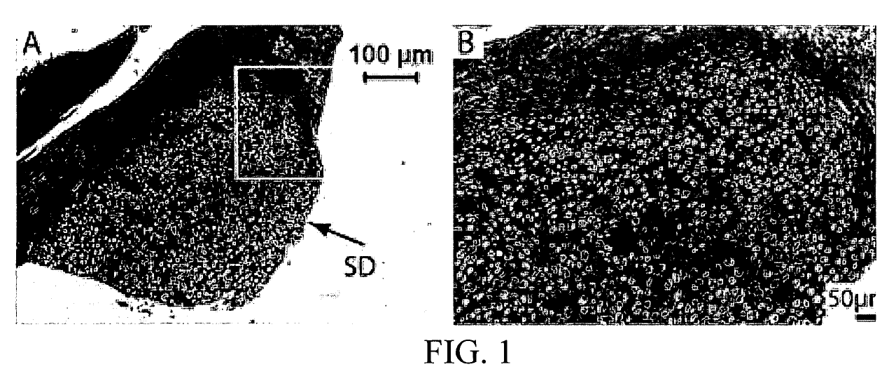

### DETAILED DESCRIPTION OF THE INVENTION

The disclosed method and compositions may be understood more readily by reference to the following detailed description of particular embodiments and the Example included therein and the previous and following description.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a polymer is disclosed and discussed and a number of modifications that can be made to a number of molecules including the polymer are discussed, each and every combination and permutation of polymer and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

Those skilled in the art will recognize, or be able to ascertain using no more than routine experimentation, many equivalents to the specific embodiments of the method and compositions described herein. Such equivalents are intended to be encompassed by the appended claims. Although many methods and materials similar or equivalent to those described herein can be used in the practice or testing of the present method and compositions, the particularly useful methods, devices, and materials are as described.

Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

### A. In Vivo Bioreactor

Disclosed herein is an in vivo method for promoting the growth of autologous tissue and its use to form corrective structures, including tissue that can be explanted to other locations in the animal. In particular, disclosed are methods and systems for (a) the site-specific regeneration of tissue, and (b) the synthesis of neo-tissue for transplantation. The disclosed methods utilize the patient's own body as the cell source, the scaffold, and the drug delivery vehicle. Methods for the generation of large volumes of bone de novo without cell transplantation and administration of exogenous growth factors by invoking a wound-healing response within a confined subperiosteal space is disclosed in U.S. Patent Publication No. 2005/0079159 A1, which is hereby disclosed herein in its entirety for the teaching of this method. This system, referred to as an “In vivo Bioreactor (IVB)” (Stevens, M. M., et al, 2005), involves the utilization of the body's own healing process to engineering autologous bone and cartilage without cell transplantation. In the IVB, the formation of fully functional bone can be achieved by simply injecting a calcium rich biomaterial such as a calcium-alginate gel within the IVB space. However, the formation of Hyaline-like cartilage within the IVB requires the local administration of an anti-angiogenic agent such as Suramin to inhibit angiogenesis (Hunziker E B, et al, 2003) and concomitant localized delivery of transforming growth factor-beta (TGF-β1) in a hyaluronic acid gel matrix (Stevens M M, et al, 2005).

The disclosed compositions and methods obviates the need for using either the anti-angiogenic agent or any growth factor molecules. Thus, the herein disclosed biocompatible hydrogel is capable of triggering chondrogenic differentiation of periosteal cells within IVB environment without the requirement of exogenous factors.

Provided herein is a method for promoting generation of cartilage (e.g., hyaline-like cartilage), comprising the step of administering in or adjacent to periosteum tissue of a subject a therapeutically effective amount of a biocompatible hydrogel. An advantage of the disclosed biocompatible hydrogel is that non-carbohydrate anti-angiogenic agents and growth factors can be substantially absent. In one aspect, the biocompatible hydrogel is biodegradable.

A “hydrogel,” as used herein, refers to a network of polymer chains that are water-soluble, sometimes found as a colloidal gel in which water is the dispersion medium. Hydrogels can be superabsorbent natural or synthetic polymers. For example, hydrogels can contain over 99% water. Hydrogels can also possess a degree of flexibility very similar to natural tissue, due to their significant water content. However, it is also understood that in one aspect, the disclosed hydrogels can comprise water or water mixed with other miscible liquids, for example, alcohols.

Hydrogels can comprise positively charged, negatively charged, and neutral hydrogels that can be saturated or unsaturated. Examples of hydrogels are TETRONICS™ and POLOXAMINES™, which are poly(oxyethylene)-poly(oxypropylene) block copolymers of ethylene diamine; polysaccharides, chitosan, poly(vinyl amines), poly(vinyl pyridine), poly(vinyl imidazole), polyethylenimine, poly-L-lysine, growth factor binding or cell adhesion molecule binding derivatives, derivatised versions of the above (e.g. polyanions, polycations, peptides, polysaccharides, lipids, nucleic acids or blends, block-copolymers or combinations of the above or copolymers of the corresponding monomers); agarose, methylcellulose, hydroxyproylmethylcellulose, xyloglucan, acetan, carrageenan, xanthan gum/ocust beangum, gelatine, collagen particularly Type 1), PLURONICS™, POLOXAMERS™, POLY(N-isopropylacrylmide) and N-isopropylacrylmide copolymers. Thus, for example, the at least one polymer can comprise a saccharide residue, an ethylene oxide residue, a propylene oxide residue, an acrylamide residue, or a blend or copolymer thereof. Thus, the at least one polymer can be agarose. The at least one polymer can be a polaxomers, or a derivative thereof. The at least one polymer can be a polyacrylamides, or a derivative thereof. The at least one polymer can be N-isopropylacrylamide (NIPAM), or a derivative thereof. The at least one polymer can be Pluronic F127, or a derivative thereof.

Also provided is a method for promoting generation of bone tissue, comprising administering an angiogenic agent in or adjacent to periosteum tissue or to the biocompatible hydrogel. In one aspect, the angiogenic agent is administered to the biocompatible hydrogel prior to administration to the tissue. In another aspect, the angiogenic agent is administered in or adjacent to periosteum tissue after chondrocytes have formed in the biocompatible hydrogel.

1. Exogenous Cells

An advantage of the herein disclosed biocompatible hydrogels is that they do not require the addition of exogenous cells, such as chondrocytes. Thus, the biocompatible hydrogel can be substantially free of exogenous cells. For example, the biocompatible hydrogel can be substantially free of exogenous chondrocytes, osteoblasts, mesenchymal stem cells (MSC), pluripotent stem cells, hematopoeitic, dermal stem cells, and myoblasts prior to implantation. As used herein, exogenous cells are cells that are added to the gel ex vivo and thus can include autologous and heterologous cells. However, it is understood that the biocompatible hydrogel can comprise endogenous, autologous cells (e.g., chondrocytes and cartilage cells) that migrate into said gel after implantation.

The biocompatible hydrogel can comprise at least about 0.1%, at least about 0.5%, at least about 1%, at least about 2%, at least about 3%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90% of the at least one polymer by weight.

2. Saccharide Polymers

The saccharide residues of the at least one polymer can be monosaccharides, disaccharides, or polysaccharides. The saccharide residues of the at least one polymer can exists in the form of a pyranose or furanose (6 or 5 member rings). The saccharide residues of the at least one polymer can be galactose sugars. The saccharide residues of the at least one polymer can comprise α1→4, β 1→3 glycosidic linkages. At least a portion of the saccharide residue of the at least one polymer can have α(1→4)-α and (1→3)-β glycosidic bond.

The saccharide residues of the at least one polymer can be lecithin, amylase, amylopectin, mannose residues, N-acetyl glucosamine, N-acetyl galactosamine, or fucose. The saccharide residues of the at least one polymer can be O-linked or N-linked glycans. The saccharide residues of the at least one polymer can be heparin sulfate, Dermatan sulfate, Chondroitin sulfate, or other proteoglycans.

The at least one polymer can be a linear polymer. The at least one polymer can be a sugar derivatized polymer. The at least one polymer can be a hyper branched star polymer. The at least one polymer can be a dendrimer. The at least one polymer can be a graft polymer.

3. Agarose

The at least one polymer can be agarose or a derivative thereof. The at least one polymer can be a carrageenan or a derivative thereof.

Agarose is an extract of agar, which consists of a mixture of agarose and agaropectin. Agar is prepared from red seaweed (Rhodophycae) and is commercially obtained from species of Gelidium and Gracilariae.

Agaropectin is a heterogeneous mixture of smaller molecules that occur in lesser amounts. Their structures are similar but slightly branched and sulfated, and they may have methyl and pyruvic acid ketal substituents. They gel poorly and may be simply removed from the excellent gelling agarose molecules by using their charge.

Agarose is a linear polymer, of molecular weight about 120,000, based on the —(13)-β-D-galactopyranose-(14)-3,6-anhydro-α-L-galactopyranose unit.

Thus, the at least one polymer can comprise poly(1→4)-3,6-anhydro-α-L-galactopyranosyl-(1→3)-β-D-galactopyranan. The at least one polymer can comprise alternating β-(1→3)-D and β-(1→4)-L linked galactose residues.

Agarose molecules have molecular weights about 120,000, The gel network of agarose contains double helices formed from left-handed threefold helices. These double helices are stabilized by the presence of water molecules bound inside the double helical cavity. Exterior hydroxyl groups allow aggregation of up to 10,000 of these helices to form suprafibers.

Thus, the at least one polymer can comprise at least two strands that form a double helix stabilized by the presence of water molecules inside the helix. The at least one polymer can comprise exterior hydroxyl groups that allow aggregation of the helices into fibers.

4. Carrageenans

The at least one polymer can be a carrageenan or a derivative thereof. Carrageenan is a collective term for polysaccharides prepared by alkaline extraction (and modification) from red seaweed (Rhodophycae), mostly of genus Chondrus, Eucheuma, Gigartina and Iridaea. Different seaweeds produce different carrageenans.

Carrageenans are linear polymers of about 25,000 galactose derivatives with regular but imprecise structures, dependent on the source and extraction conditions. The major differences between agarose and carrageenans being the presence of L-3,6-anhydro-β-galactopyranose rather than D-3,6-anhydro-α-galactopyranose units and the lack of sulfate groups.

The idealized structure of κ-carrageenan (kappa-carrageenan) is: —(1→3)-β-D-galactopyranose-4-sulfate-(1→4)-3,6-anhydro-α-D-galactopyranose-(1→3)-—

κ-carrageenan is produced by alkaline elimination from μ-carrageenan isolated mostly from the tropical seaweed Kappaphycus alvarezii (also known as Eucheuma cottonii). The experimental charge/dimer is 1.03 rather than 1.0 with 0.82 molecules of anhydrogalactose rather than one.

The idealized structure of ι-carrageenan (iota-carrageenan) is: —(1→3)-β-D-galactopyranose-4-sulfate-(1→4)-3,6-anhydro-α-D-galactopyranose-2-sulfate-(1→3)—

ι-carrageenan is produced by alkaline elimination from v-carrageenan isolated mostly from the Philippines seaweed Eucheuma denticulatum (also called Spinosum). The experimental charge/dimer is 1.49 rather than 2.0 with 0.59 molecules of anhydrogalactose rather than one. The three-dimensional structure of the ι-carrageenan double helix has been determined [247] as forming a half-staggered, parallel, threefold, right-handed double helix, stabilized by interchain O2—H . . . O—5 and O6—H . . . O-2 hydrogen bonds between the β-D-galactopyranose-4-sulfate units.

The idealized structure of λ-carrageenan (lambda-carrageenan) is: —(1→3)-β-D-galactopyranose-2-sulfate-(1→4)-α-D-galactopyranose-2,6-disulfate-(1→3)

λ-carrageenan (isolated mainly from Gigartina pistillata or Chondrus crispus) is converted into θ-carrageenan (theta-carrageenan) by alkaline elimination, but at a much slower rate than causes the production of ι-carrageenan and κ-carrageenan. The experimental charge/dimer is 2.09 rather than 3.0 with 0.16 molecules of anhydrogalactose rather than zero.

5. Structure

Also as disclosed herein, the at least one polymer can comprise one or more saccharide residues having the structure:

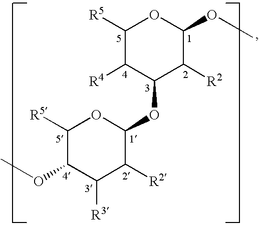

wherein R², R²′, and R³′, are, independently, hydrogen, hydroxyl, alkoxyl, alkylether, amine, or amide;

wherein R⁴ is hydrogen, hydroxyl, alkoxyl, alkylether, amine, or amide; and

wherein R⁵ and R⁵′ are, independently, hydrogen, hydroxyl, alkoxyl, alkyl, hydroxymethylene, alkylether, amine, or amide.

Thus, the at least one polymer can comprise one or more saccharide residues having the structure:

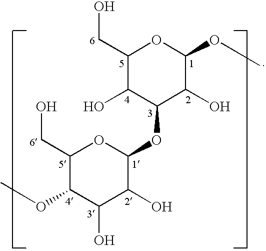

The at least one polymer can also comprise one or more saccharide residues having the structure:

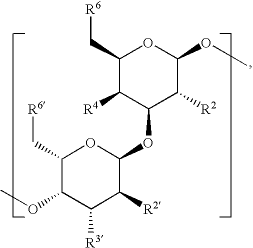

wherein R², R² ′, R⁴′, R³′, R^{6,} and R⁶′ can independently be hydrogen, hydroxyl, or alkoxyl.

The polymer can also comprise one or more saccharide residues having the structure:

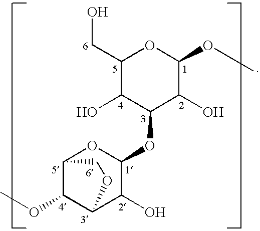

The polymer can also comprise one or more saccharide residues having the structure:

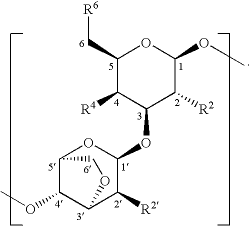

The polymer can also comprise one or more saccharide residues having the structure:

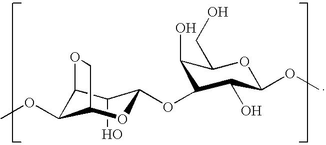

6. Anti-angiogenic Agents

Angiogenesis has been shown to impede the repair of articular cartilage defects. To overcome this obstacle, sustained levels of anti-angiogenic agents have been used during chondrogenic treatments (Hunziker E B, et al, 2003). An advantage of the herein disclosed biocompatible hydrogels is that they do not require the addition of anti-angiogenic agents in order to stimulate chondrogenesis from periosteal tissue. For example, endothelial cells are not capable of degrading agarose. Hence, agarose is an intrinsically antiangiogenic material (Helmlinger G, et al. 1997).

However, anti-angiogenic compositions, while not required, can be used or added in some aspects of the disclosed methods.

Thus, in some aspect of the provided methods, anti-angiogenic agents are substantially absent from the biocompatible hydrogel. In other aspects of the disclosed methods, a carbohydrate polymer disclosed for use in the provided biocompatible hydrogel is also considered anti-angiogenic. In other aspects of the disclosed methods, the provided biocompatible hydrogel does not comprise a carbohydrate polymer that is anti-angiogenic. In still other aspects of the disclosed methods, the biocompatible hydrogel is substantially free of non-carbohydrate, anti-angiogenic agents.

Examples of anti-angiogenic agents include, but are not limited to, endostatin, angiostatin, TNP-470, angiozyme, anti-VEGF antibody (Avastin®; bevacizumab), VEGF receptor tyrosine kinase inhibitor, benefin, BMS275291, bryostatin-I (SC339555), CAI, CM101, combretastatin, dexrazoxane (ICRF187), DMXAA, EMD 121974, flavopiridol, GTE, heparin/cortisone, hydrocortisone, IM862, interferon-α, interlukin-12, BMP inhibitors (e.g., noggin), TGF-beta family inhibitors, inhibitors of matrix metalloproteinases such as marimastat, metaret, metastat, MMI-270, neovastat, octreotide (somatostatin), paclitaxel (taxol), purlytin, PTK787, cartilage extract, squalarnine, suradista (FCE26644), SU101, SU5416, SU6668, suramin, tamoxifen (nolvadex), tetrathiomolybdate, thalidomide, vitaxin and xeloda (capecitabine), cycloogenase, platelet factor 4 (PF-4), an N-terminally truncated proteolytically cleaved PF-4 fragment, a 16 kDa N-terminal fragment of human prolactin, smaller protein fragments of fibronectin, murine epidermal growth factor, and thrombospondin.

The biocompatible hydrogel can be substantially free of sulfated oligosaccharides. Thus, the biocompatible hydrogel can be substantially free of sulfated cyclic sugars. Thus, the biocompatible hydrogel can be substantially free of sulfated cyclodextrins.

7. Growth Factors

An advantage of the herein disclosed biocompatible hydrogels is that they do not require the addition of exogenous growth factors in order to stimulate chondrogenesis from periosteal tissue. Thus, growth factors can also be substantially absent from the biocompatible hydrogel. As used herein, a “growth factor” includes any soluble factor that regulates or mediates cell proliferation, cell differentiation, tissue regeneration, cell attraction, wound repair and/or any developmental or proliferative process. For example, fibroblast growth factor-2 (FGF-2), fibroblast growth factor-1 (FGF-1), epidermal growth factor (EGF), heparin binding growth factor (HBGF), Placental Growth Factor (PlGF), vascular endothelial growth factor (VEGF), transforming growth factor-alpha (TGF-α), transforming growth factor-beta (TGF-β), insulin-like growth factor (IGF-I, IGF-II), platelet derived growth factor (PDGF), leukemia inhibitory factor (LIF), and platelet rich plasma (PRP) can be substantially absent from the biocompatible hydrogel.

8. Pharmaceutically Active Agents

The biocompatible hydrogel can further comprise at least one pharmaceutically active agent. As used herein, the term “pharmaceutically active agent” includes a “drug” and means a molecule, group of molecules, complex or substance administered to an organism for diagnostic, therapeutic, preventative medical, or veterinary purposes. This term includes human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term may also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a bioactive effect, for example deoxyribonucleic acid (DNA), ribonucleic acid (RNA), or mixtures or combinations thereof, including, for example, DNA nanoplexes, antisense molecules, aptamers, ribozymes, triplex forming molecules, RNAi, and external guide sequences. Pharmaceutically active agents include the herein disclosed categories and specific examples. It is not intended that the category be limited by the specific examples. Those of ordinary skill in the art will recognize also numerous other compounds that fall within the categories and that are useful according to the invention.

Examples include a radiosensitizer, a steroid, a xanthine, an anti-inflammatory agent, an analgesic agent, an anticoagulant agent, an antiplatelet agent, a sedative, an antineoplastic agent, an antimicrobial agent, an antifungal agent, a protein, or a nucleic acid. Thus, the pharmaceutically active agent can be coumarin, albumin, steroids such as betamethasone, dexamethasone, methylprednisolone, prednisolone, prednisone, triamcinolone, budesonide, hydrocortisone, and pharmaceutically acceptable hydrocortisone derivatives; antiinflammatory agents, including antiasthmatic antiinflammatory agents, antiarthritis antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetominophen, ibuprofen, ketoprofen and piroxicam; analgesic agents such as salicylates; anticoagulant and antiplatelet agents such as coumadin, warfarin, acetylsalicylic acid, and ticlopidine; sedatives such as benzodiazapines and barbiturates; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; antimicrobial agents such as penicillins, cephalosporins, and macrolides, antifungal agents such as imidazolic and triazolic derivatives; and nucleic acids such as DNA sequences encoding for biological proteins, and antisense oligonucleotides.

Thus, the at least one pharmaceutically active agent can be selected from hydrocortisone, steroidal anti-inflammatory drugs, non-steroidal anti-inflammatory drugs (NSAID's), anesthetics, analgesics, and mixtures thereof.

9. Additional Polymers

The biocompatible hydrogel can further comprise at least one other biocompatible polymer. For example, the at least one other biocompatible polymer can comprise hyaluronic acid, heparin, a heparin fragment, glycosaminoglycans, glycosylated proteins (proteoglycans), glycosylated non-degradable and degradable synthetic polymers, polymers with sugar residues, or a combinations thereof.

The at least one other biocompatible polymer can comprise a self-assemble peptide. Certain peptides are able to self-assemble into stable hydrogels at low (0.1-1%) peptide concentrations (Zhang S, et al, 1993; Zhang S, et al, 1995; Holmes T C, et al, 2000). Such self-assembling peptides are characterized by amino acid sequences of alternating hydrophobic and hydrophilic side groups. Sequences of charged amino acid residues include alternating positive and negative charges (Zhang S, et al, 1993; Zhang S, et al, 1995; Holmes T C, et al, 2000). Self-assembling peptides form stable β-sheet structures when dissolved in deionized water. Exposure to electrolyte solution initiates β-sheet assembly into interweaving nanofibers. Such self-assembly occurs rapidly when the ionic strength of the peptide solution exceeds a certain threshold, or the pH is such that the net charge of the peptide molecules is near zero (Caplan M R, et al, 2000). Intermediate steps of self-assembly have been investigated by observing relatively slow nanofiber formation and subsequent network assembly in deionized water, without triggering rapid self-assembly by the addition of electrolytes (Marini D M, et al, 2002). The self-assembling peptide hydrogel contains unique features for a tissue engineering polymer scaffold. The nanofiber structure is almost 3 orders of magnitude smaller than most polymer microfibers and presents a unique polymer structure with which cells may interact. In addition, peptide sequences may be designed for specific cell-matrix interactions that influence cell differentiation and tissue formation (Holmes T C., 2002). For example, self-assembling peptide KLD-12 hydrogel has been studied as a 3D scaffold for encapsulation of chondrocytes (Kisiday et al, 2002).

The biocompatible hydrogel can further comprise block copolymers such as PLURONICS™ (also known as POLOXAMERS™), which are poly(oxyethylene)-poly(oxypropylene) block polymers solidified by changes in temperature, or TETRONICS™ (also known as POLOXAMINES™), which are poly(oxyethylene)-poly(oxypropylene) block polymers of ethylene diamine solidified by changes in pH.

The biocompatible hydrogel can also include a shield to exclude in-growth of unwanted tissue phenotypes. Although it is generally beneficial to place the largest possible proportion of the surface area of the scaffold in direct contact with, or adjacent to, target tissue (mature or immature), there may be tissue types in the vicinity of the implant that will be detrimental to the formation of target tissue (e.g., tissue types that populate the scaffold but that are not, or do not produce, target tissue). A shield can be used to reduce or prevent these unwanted cells from infiltrating the scaffold. The shield can be placed around the part of the scaffold adjacent to cells of the unwanted tissue type. The shield should be too dense to allow the passage of cells, but porous enough to permit nutrients to reach the cells associated with the scaffold (and allow waste products to diffuse away). The shield may: include a non-porous barrier that allows moisture, but not cells, to reach the scaffold (e.g. by diffusion through the barrier); be impermeable to both fluid and cells, allowing neither to reach the scaffold; or it may be porous, allowing fluids and nutrients to reach cells that have moved into the scaffold from unshielded portion, but not allowing the passage of cells. The shield can be removed from the graft prior to the time the graft is implanted (i.e., prior to the time the graft is used to treat a patient with a damaged target tissue). For example the shield can have a pore size of less than 0.85 ∞m. A high moisture transmission rate polymer that lacks physical perforation may be employed, for example “HPU 25”, a copolymer of Desmodur W (dicyclohexylmethane-4,4′-diisocyanate), polyethylene glycol, ethylene glycol and water to give a copoly(ester-urea-urethane) which is an elastomer with very high moisture transmission rate and which may be cast from solution to give a conformable film

10. Oxygen Permeability

The biocompatible hydrogel of the provided method can have a low oxygen permeability and diffusion. Oxygen permeability refers to the rate at which oxygen will pass through a material under specified conditions and specimen geometry. Thus, the biocompatible hydrogel can be hypoxic. For example, Agarose has been shown to mimic the tumor microenvironment by limiting the diffusion of metabolites and by reducing the oxygen concentration to levels similar to those found in solid tumors (Gunther M, et al. 2006)

The effective diffusion coefficient of oxygen, IDₑ, can be determined in biocompatible hydrogels using standard techniques. See Hulst, et al. 1989 and McCabe M, et al. 1975, which are hereby incorporated herein by reference for the teaching of methods of determining oxygen diffusion. These methods have demonstrated, for example, a decreasing IDₑ for both agar and agarose at increasing gel concentration. In case of calcium alginate and gellan gum, a maximum in IDₑ at the intermediate gel concentration is observed. This phenomenon can be due to a changing gelpore structure at increasing gel concentrations. For example, the IDₑ of oxygen in calcium alginate, κ-carrageenan and gellan gum can vary from 1.5×10⁻⁹ to 2.1×10⁻⁹ m²s⁻¹ in the gel concentration range of 0.5 to 5% (w/v).

Thus, in one aspect, the IDₑ of the biocompatible hydrogel is less than about 1.5×10⁻⁷ m²s⁻¹, less than about 1×10⁻⁸ m²s⁻¹, less than about 1×10⁻⁹ m²s⁻¹, less than about 1.5×10⁻⁹ m²s⁻¹, less than about 1.4×10⁻⁹ m²s⁻¹, less than about 1.3×10⁻⁹ m²s⁻¹, less than about 1.2×10⁻⁹ m²s⁻¹, less than about 1.1×10⁻⁹ m²s⁻¹, less than about 1×10⁻⁹ m²s⁻¹, less than about 9×10⁻¹⁰ m²s⁻¹, less than about 8×10⁻¹⁰ m²s⁻¹, less than about 7×10⁻¹⁰ m²s⁻¹, less than about 6×10⁻¹⁰ m²s⁻¹, less than about 5×10⁻¹⁰ m²s⁻¹, less than about 4×10⁻¹⁰ m²s⁻¹, less than about 3×10⁻¹⁰ m²s⁻¹, less than about 2×10⁻¹⁰ m²s⁻¹, or less than about 1×10⁻¹⁰ m²s⁻¹.

Thus, the IDₑ of the biocompatible hydrogel can be from at least about 0 to 1.5×10⁻⁷ m²s⁻¹, at least about 0 to 1.5×10⁻⁷ m²s⁻¹, at least about 0 to 1×10⁻⁸ m²s⁻¹, at least about 0 to 1×10⁻⁹ m²s⁻¹, at least about 0 to 1.5×10⁻⁹ m²s⁻¹ , at least about 0 to 1.4×10⁻⁹ m²s⁻¹, at least about 0 to 1.3×10⁻⁹ m²s⁻¹, at least about 0 to 1.2×10⁻⁹ m²s⁻¹, at least about 0 to 1.1×10⁻⁹ m²s⁻¹, at least about 0 to 1×20⁻⁹ m²s⁻¹, at least about 0 to 9×10⁻¹⁰ m²s⁻¹, at least about 0 to 8×10 ⁻¹⁰ m²s⁻¹, at least about 0 to 7×10⁻¹⁰ m²s⁻¹, at least about 0 to 6×10⁻¹⁰ m²s⁻¹, at least about 0 to 5×10⁻¹⁰ m²s⁻¹, at least about 0 to 4×10⁻¹⁰ m²s⁻¹, at least about 0 to 3×10⁻¹⁰ m²s⁻¹, at least about 0 to 2×10⁻¹⁰ m²s⁻¹, or at least about 0 to 1×10⁻¹⁰ m²s⁻¹.

Thus, the IDₑ of the biocompatible hydrogel can be from about 1×10⁻¹⁰ to 1.5×10⁻⁷ m²s⁻¹, about 1×10⁻¹⁰ to 1.5×10⁻⁷ m²s⁻¹, about 1×10⁻¹⁰ to 1×10⁻⁸ m²s⁻¹, about 1×10⁻¹⁰ to 1×10⁻⁹ m²s⁻¹, about 1×10⁻¹⁰ to 1.5×10⁻⁹ m²s⁻¹, about 1×10⁻¹⁰ to 1.4×10⁻⁹ m²s⁻¹, about 1×10⁻¹⁰ to 1.3×10⁻⁹ m²s⁻¹, about 1×10⁻¹⁰ to 1.2×10⁻⁹ m²s⁻¹, about 1×10⁻¹⁰ to 1.1×10⁻⁹ m²s⁻¹, about 1×10⁻¹⁰ to 1×10⁻¹⁰ m²s⁻¹, about 1×10⁻¹⁰ to 9×10⁻¹⁰ m²s⁻¹, about 1×10⁻¹⁰ to 8×10⁻¹⁰ m²s⁻¹, about 1×10 ⁻¹⁰ 7×10⁻¹⁰ m²s⁻¹, about 1×10⁻¹⁰ to 6×10⁻¹⁰ m²s⁻¹, about 1×10⁻¹⁰ to 5×10⁻¹⁰ m²s⁻¹, about 1×10⁻¹⁰ to 4×10⁻¹⁰ m²s⁻¹, about 1×⁻¹⁰ to 3×10⁻¹⁰ m²s⁻¹, or about 1×10⁻¹⁰ to 2×10−19 m²s⁻¹.

Oxygen diffusion can be a function of the porosity of the gel. As the biocompatible hydrogel is at least partially porous, it allows tissue in-growth. When the biocompatible hydrogel contains interconnected pores that are evenly distributed, cells can infiltrate essentially all areas of the scaffold during the period of implantation. The pore diameter is determined by, inter alia, the need for adequate surface area for tissue in-growth and adequate space for nutrients and growth factors to reach the cells. The percentage open volume of the scaffold is selected by balancing the needs for “open” volume, which allows and adequate number of cells and sufficient nutrients to permeate quickly through the structure and desirable oxygen diffusion. Thus, the percentage open volume can be less than about 90%, less than about 80%, less than about 70%, less than about 60%, less than about 50%, less than about 40%, less than about 30%, less than about 20%, or less than about 10%.

Thus, in another aspect, the average pore size in the biocompatible hydrogel is less than about 10 nm, less than about 50 nm, less than about 100 nm, less than about 200 nm, less than about 300 nm, less than about 400 nm, less than about 500 nm, less than about 600 nm, less than about 700 nm, less than about 800 nm, less than about 900 nm, or less than about 1000 nm. Thus, the average pore size can be from about 1 μm to 10 nm, from about 1 μm to 50 nm, from about 1 μm to 100 nm, from about 1 μm to 200 nm, from about 1 μm to 300 nm, from about 1 μm to 400 nm, from about 1 μm to 500 nm, from about 1 μm to 600 nm, from about 1 μm to 700 nm, from about 1 μm to 800 nm, from about 1 μm to 900 nm, or from about 1 μm to 1000 nm.

11. Modulus

The biocompatible hydrogel of the provided method can have a high elastic modulus. For example, the modulus can be greater than 0.001, 0.05, 0.01, 0.1, 0.2, 0.3, 0.4, 0.5, 0.6, 0.7, 0.8, 0.9, 1, 2, 3, 4, 5, 6, 7, 8, 9, 10, 15, 20, 50 megapascals. Compositions such as sodium alginate that can be used to increase the modulus of the biocompatible hydrogel are known in the art.

In one aspect, the elastic modulus is determined in part by the concentration of the biocompatible hydrogel, such as agarose. Thus, as an example, wherein the biocompatible hydrogel is agarose, the concentration of agarose can be at least about 1%, 2%, 3%, 4%, 5%, 6%, 7%, 8%, 9%, 10%, 11%, 12%, 13%, 14%, 15%, 16%, 17%, 18%, 19%, 20%, or higher.

An elastic modulus, or modulus of elasticity, is the mathematical description of an object or substance's tendency to be deformed when a force is applied to it. The elastic modulus of an object is defined as the slope of its stress-strain curve:

λ=stress/strain

wherein λ is the elastic modulus; stress is the force causing the deformation divided by the area to which the force is applied; and strain is the ratio of the change caused by the stress to the original state of the object. Because stress is measured in pascals and strain is a unitless ratio, the units of λ are therefore pascals as well.

The concept of a constant elastic modulus is dependent on the assumption that the stress-strain curve is always linear. In reality, the curve is only linear within certain limits, because an object stretched or compressed too far will break, and an object under high pressure may undergo processes that will affect the stress-strain curve, such as chemical reactions or buckling. Thus, there are three primary elastic moduli, each describing a different kind of deformation. They are Young's modulus, modulus of rigidity, and bulk modulus. Young's modulus (E) describes tensile elasticity, or the tendency of an object to deform along an axis when opposing forces are applied along that axis; it is defined as the ratio of tensile stress to tensile strain. Because all other elastic moduli can be derived from Young's modulus, it is often referred to simply as the elastic modulus. Young's modulus is a mathematical consequence of the Pauli exclusion principle. The shear modulus or modulus of rigidity (G) describes an object's tendency to shear (the deformation of shape at constant volume) when acted upon by opposing forces; it is defined as shear stress over shear strain. The shear modulus is part of the derivation of viscosity. The bulk modulus (K) describes volumetric elasticity, or the tendency of an object's volume to deform when under pressure; it is defined as volumetric stress over volumetric strain, and is the inverse of compressibility. The bulk modulus is an extension of Young's modulus to three dimensions.

In another aspect, the biocompatible hydrogel has a stress-field high enough that the hydrogel is anti-angiogenic in nature, since the endothelial cell can not overcome the stress field. Thus, for example, the stress-field of the biocompatible hydrogel can be at least about 80, 90, 95, 100, 105, 110, 115, or 120 mm Hg. Thus, the stress-field of the biocompatible hydrogel can from about 80 to 120 mm Hg, 90 to 120 mm Hg, 95 to 120 mm Hg, 100 to 120 mm Hg, 105 to 120 mm Hg, or 110 to 120 mm Hg.

12. Immunogenicity

The biocompatible hydrogel can induce an immune response in the subject. For example, the biocompatible hydrogel can induce the local production of cytokines, wherein at least one of the cytokines stimulates chondrogenic differentiation of the periosteal cells.

The induction of immune responses can be divided into cell-mediated and antibody-mediated immune responses. The CD4⁺ T helper (Th) cells involved in these two pathways are of Th1-type for cell-mediated immunity (CMI), which contribute to clearance of virally infected cells and CD4⁺ Th2-type which are involved in antibody-mediated immunity. The role of these two major Th cell subsets for induction of specific immunity is in large part determined by the cytokines produced, where Th1 cells secrete IL-2, IFN-γ and LT-α, while Th2 cells secrete IL-4, IL-5, IL-6, IL-10 and IL-13.

Thus, in one aspect, the biocompatible hydrogel induces the local production of IL-2, IFN-γ, LT-α, IL-4, IL-5, IL-6, IL-10, IL-13, or a combination thereof, wherein at least one of the cytokines stimulates chondrogenic differentiation of the periosteal cells.

13. Artificial Space

In one aspect, the biocompatible hydrogel is administered into the space between the periosteum and the bone without substantially disturbing the periosteum membrane. For example, the biocompatible hydrogel can be administered through a small hole, cut, or tear such that few if any cells other than the cells recruited from the periosteum can enter this space.

The provided method can further comprise the step of creating an artificial space or environment in an organ or cavity of the subject prior to the administration step. In a preferred aspect, the artificial space is created in such a way as to minimize invasion of the space by cells other than those recruited from the periosteum. For example, a tissue retractor can be used to generate the artificial space. The retractor can selectively move appropriate tissue out of the way to form the space abutting a mesenchymal portion of the tissue or the space in the periosteum. For instance, examples of retractors useful in the disclosed methods include a fluid-operated portion such as a balloon or bladder to retract tissue, not merely to work in or dilate an existing opening, as for example an angioscope does. The fluid-filled portion of the retractor can be flexible and, thus, have no sharp edges that might injure tissue being moved by the retractor. The soft material of the fluid-filled portion, to an extent desired, can conform to the tissue confines, and the exact pressure can be monitored so as not to damage tissue.

The retractor can have a portion which is expandable upon the introduction of fluid under pressure. The expandable portion can be made of a material strong enough, and can be inflated to enough pressure, to spread adjoining tissues within the body. In the case of use with tissue such as the periosteum, the expandable portion can have sufficient rigidity such that it does deform during the expansion process, e.g., have edges which “leak out” from the site to be expanded.

The bladder can be pressurized with air or with water or another fluid. The fluid used in the bladder must be safe in case it accidentally escapes into the body. Thus, besides air, such other fluids as dextrose water, normal saline, CO₂, and N₂ are safe. The pressure in the bladder can be monitored and regulated to keep the force exerted by the retractor at a safe level for the tissue to prevent tissue necrosis. The retractor can exert a pressure on the tissues of as high as the mean diastolic pressure of 100 mm of mercury, or higher for shorter periods of time, while still being safely controlled. The bladder may be of such materials such as KEVLAR®, MYLAR®, OR DYNEEMA®, which may be reinforced with stainless steel, nylon, or other fiber to prevent puncturing and to provide structural shape and support as desired. Such materials are strong enough to hold the necessary fluid pressure of about several pounds or up to about 500 mg Hg or more and exert the needed force on the tissue to be moved.

The artificial space can be created by hydraulic elevation. A method for harvesting periosteum by hydraulic elevation is provided in Marini R P, Stevens M M, Langer R, Shastri V P. Hydraulic elevation of the periosteum: a novel technique for periosteal harvest. J Invest Surg 17 (4), 229 (2004), which is hereby incorporated herein by reference for the teaching of this method and its application for creating an artificial space.

Ultrasonic or other cutting or ablative devices can also be used to remove surrounding tissue to permit the expansion of the artificial space.

The area in which the artificial space is to be created can be treated with an agent to partially degrade the connective tissue at the site, freeing cells to promote formation of the space and/or promote migration of cells into the space. For example, the area can be treated with an agent selected from the group consisting of trypsin, chymotrypsin, collagenase, elastase, hyaluronidase, pronase and chondroitinase.

Stents and other barriers can be used to help hold the shape or volume of the expanded area. External pressure can be applied to the matrix, such as by application of a pressure bandage or inflated air bladder in the proximal to the cavity.

14. Implantation

The biocompatible hydrogel can be deformed as it is implanted, allowing implantation through a small opening in the patient or through a cannula or instrument inserted into a small opening in the patient. After implantation, the biocompatible hydrogel can expand into its desired shape and orientation. Thus, it is desired that the disclosed biocompatible hydrogel be at least marginally flexible, compressible, and/or resilient. For example, the elastic modulus of the biocompatible hydrogel can be less than 500, 400, 300, 200, 100, 50, 40, 30, 20, 10, 9, 8, 7, 6, 5, 4, 3, 2, or 1 megapascals.

The biocompatible hydrogel can be warmed to melting temperature prior to implantation. In this embodiment, gelation of the melted biocompatible hydrogel can be enhanced by cooling. For example, chilled (e.g., 5° C.) sterile 0.9% NaCl can be administered.

15. Harvesting

The provided method can involve harvesting progenitor cells (e.g., chondrocytes) from the artificial space, or alternatively, the cells can be caused to mature to a cell or tissue phenotype of the desired functional and histological end-point (e.g., cartilage), then harvested. Cells/tissue isolated by the disclosed method can be further manipulated ex vivo, e.g. further expanded or differentiated. The cells/tissue can be banked, e.g, cryogenically preserved, or used for transplantation.

Thus, the provided method can further comprise the step of harvesting chondrocytes from the artificial space or environment after the administration step. The provided method can further comprise the step of harvesting cartilage from the artificial space or environment after the administration step. In one aspect, the cartilage is hyaline-like cartilage. The provided method can further comprise the step of harvesting chondrocytes from the biocompatible hydrogel after the administration step. The provided method can further comprise the step of re-introducing the harvested chondrocytes and/or cartilage into the subject.

Also provided herein is a tissue graft comprising chondrocytes produced and harvested using the herein disclosed methods. The tissue graft can be produced by administering harvested chondrocytes to a biocompatible scaffold, such as those disclosed in, for example, U.S. Pat. No. 7,108,721, which is hereby incorporated herein by reference for the teaching of tissue grafts. Alternatively, the herein disclosed biocompatible hydrogel, such as agarose, can further comprise a biocompatible scaffold suitable for explantation. In this aspect, the provided tissue graft comprises the explanted biocompatible hydrogel comprising endogenously produced chondrocytes.

### B. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for promoting generation of tissue in vivo, comprising a tissue retractor for generating the artificial space; a biocompatible hydrogel disclosed herein; and (optionally) an agent to partially degrade the connective tissue at the site, freeing cells to promote formation of the space and/or promote migration of cells into the space. Also disclosed is a kit comprising a biocompatible hydrogel disclosed herein, a means for warming said gel to a melting temperature, and a means for the delivery of the melted gel to a site in a subject.

### C. Uses

The disclosed methods and compositions are applicable to numerous areas including, but not limited to growth of autologous cartilage and bone tissue for in situ repair or autologous transplant. Other uses are disclosed, apparent from the disclosure, and/or will be understood by those in the art.

### D. Saccharide Structures

The terms “saccharide” and “carbohydrate” embrace a wide variety of chemical compounds, such as monosaccharides, disaccharides, oligosaccharides and polysaccharides. Oligosaccharides are chains composed of saccharide units, which are alternatively known as sugars. These saccharide units can be arranged in any order and the linkage between two saccharide units can occur in many of approximately ten different ways. As a result, the number of different possible stereoisomeric oligosaccharide chains is enormous.

1. Selection

In various aspects, a polymer of saccharide residues can comprise any naturally occurring oligosaccharide or polysaccharide known to those of skill in the art. That is, in one aspect, a naturally occurring oligosaccharide or polysaccharide can be selected and employed as a polymer of saccharide residues. In mammalian systems, naturally occurring oligosaccharide or polysaccharide typically comprise one or more of eight monosaccharides activated in the form of nucleoside mono- and diphosphate sugars provide the building blocks for most oligosaccharides: UDP-Glc, UDP-GlcUA, UDP-GlcNAC, UDP-Gal, UDP-GalNAc, GGP-Man, GDP-Fuc and CMP-NeuAc. These are the intermediates of the Leloir pathway. A much larger number of sugars (e.g., xylose, arabinose) and oligosaccharides are present in microorganisms and plants.

In general, carbohydrate, oligosaccharide, polysaccharide, and sugar each refer to chemical compounds that contain oxygen, hydrogen, and carbon atoms. These compounds can also be optionally substituted and can also contain other elements such as sulfur or nitrogen, but these are usually minor components. Typically, carbohydrates consist of monosaccharide sugars, of varying chain lengths, that have the general chemical formula Cₙ(H₂O)ₙ or are derivatives of such. For an oligosaccharide, the smallest value for “n” is 2. For a polysaccharide, the smallest value for “n” is 3. A 3-carbon sugar is referred to as a triose, whereas a 6-carbon sugar is called a hexose. Carbohydrates can be classified by the number of constituent sugar units: monosaccharides (such as glucose and fructose), disaccharides (such as sucrose and lactose), oligosaccharides, and polysaccharides (such as starch, glycogen, and cellulose).

The simplest carbohydrates are monosaccharides, which are small straight-chain aldehydes and ketones with many hydroxyl groups added, usually one on each carbon except the functional group. Other carbohydrates are composed of monosaccharide units and break down under hydrolysis. These may be classified as disaccharides, oligosaccharides, or polysaccharides, depending on whether they have two, several, or many monosaccharide units.

Monosaccharides may be divided into aldoses, which have an aldehyde group on the first carbon atom, and ketoses, which typically have a ketone group on the second. They may also be divided into trioses, tetroses, pentoses, hexoses, and so forth, depending on how many carbon atoms they contain. For instance, glucose is an aldohexose, fructose is a ketohexose, and ribose is an aldopentose.

Disaccharides are composed of two monosaccharide units bound together by a covalent glycosidic bond. The binding between the two sugars results in the loss of a hydrogen atom (H) from one molecule and a hydroxyl group (OH) from the other.

Common disaccharides include sucrose (cane or beet sugar—made from one glucose and one fructose), lactose (milk sugar—made from one glucose and one galactose), maltose (made of two glucoses linked alpha-1,4) and cellobiose (made of two glucoses linked beta-1,4). The formula of these disaccharides is C₁₂H₂₂O₁₁. Other examples of disaccharides include trehalose, chitobiose, laminaribiose, kojibiose, and xylobiose.

Oligosaccharides and polysaccharides are composed of longer chains of monosaccharide or disaccharide units bound together by glycosidic bonds. The distinction between the two is based upon the number of monosaccharide units present in the chain. Oligosaccharides typically contain between two and nine monosaccharide units, and polysaccharides typically contain greater than ten monosaccharide units. Examples of oligosaccharides include the disaccharides mentioned above, the trisaccharide raffinose and the tetrasaccharide stachyose.

Oligosaccharides are found as a common form of protein posttranslational modification. Such posttranslational modifications include the Lewis oligosaccharides responsible for blood group incompatibilities, the alpha-Gal epitope responsible for hyperacute rejection in xenotransplanation, and O-GlcNAc modifications.

Polysaccharides represent an important class of biological polymer. Examples include starch, cellulose, chitin, glycogen, callose, laminarin, xylan, and galactomannan.

In a further aspect, compounds containing other elements can be counted as carbohydrates (e.g., glucosamine and chitin, which contain nitrogen).

2. Synthesis

The skilled artisan can select among the numerous techniques for the synthesis of carbohydrates that have been developed. Some of these techniques suffer the difficulty of requiring selective protection and deprotection. Organic synthesis of oligosaccharides is further hampered by the lability of many glycosidic bonds, difficulties in achieving regioselective sugar coupling, and generally low synthetic yields. These difficulties, together with the difficulties of isolating and purifying carbohydrates and of analyzing their structures, has made this area of chemistry a most demanding one.

For certain applications, enzymes have been targeted for use in organic synthesis as one alternative to more traditional techniques. For example, enzymes have been used as catalysts in organic synthesis; the value of synthetic enzymatic reactions in such areas as rate acceleration and stereoselectivity has been demonstrated. Additionally, techniques are now available for low cost production of some enzymes and for alteration of their properties. The use of enzymes as catalysts for the synthesis of carbohydrates has been proposed, but to date enzyme-based techniques have not been found which are useful for the general synthesis of oligosaccharides and other complex carbohydrates in significant amounts. A major limiting factor to the use of enzymes as catalysts in carbohydrate synthesis is a limited availability of the broad range of enzymes required to accomplish carbohydrate synthesis. See Toone et al., Tetrahedron Reports (1990)(45)17:5365-5422.

In mammalian systems, eight monosaccharides activated in the form of nucleoside mono- and diphosphate sugars provide the building blocks for most oligosaccharides: UDP-Glc, UDP-GlcUA, UDP-GlcNAC, UDP-Gal, UDP-GalNAc, GGP-Man, GDP-Fuc and CMP-NeuAc. These are the intermediates of the Leloir pathway. A much larger number of sugars (e.g., xylose, arabinose) and oligosaccharides are present in microorganisms and plants.

Two groups of enzymes are associated with the in vivo synthesis of oligosaccharides. The enzymes of the Leloir pathway comprise the largest group. These enzymes transfer sugars activated as sugar nucleoside phosphates to a growing oligosaccharide chain. Non-Leloir pathway enzymes transfer carbohydrate units activated as sugar phosphates, but not as sugar nucleoside phosphates.

Two strategies have been proposed for the enzyme-catalyzed in vitro synthesis of oligosaccharides. See Toone et al., supra. The first strategy proposes to use glycosyltransferases. The second proposes to use glycosidases or glycosyl hydrolases. Glycosyltransferases catalyze the addition of activated sugars, in a stepwise fashion, to a protein or lipid or to the non-reducing end of a growing oligosaccharide. A very large number of glycosyltransferases appear to be necessary to synthesize carbohydrates. Each NDP-sugar residue requires a distinct class of glycosyltransferases and each of the more than one hundred glycosyltransferases identified to date appears to catalyze the formation of a unique glycidic linkage.

Enzymes of the Leloir pathway have begun to find application to the synthesis of oligosaccharides. Two elements are required for the success of such an approach. The sugar nucleoside phosphate must be available at practical cost and the glycosyltransferase must be available. The first issue is resolved for most common NDP-sugars, including those important in mammalian biosynthesis. The problem in this technology however resides with the second issue. To date, a relatively small number of glycosyltransferases are available.

In various aspects, any technique for preparing an oligosaccharide or polysaccharide that is known to those of skill in the art can be used to prepare one or more of the disclosed polymers and/or the disclosed saccharide residues. That is, at least one polymer of saccharide residues can be provided using such methods. For example, one or more techniques disclosed in U.S. Pat. Nos. 3,666,627; 3,930,950; 4,150,116; 4,184,917; 4,219,571; 4,250,262; 4,264,227; 4,359,531; 4,386,158; 4,451,566; 4,537,763; 4,557,927; 4,569,909; 4,590,160; 4,594,321; 4,617,269; 4,621,137; 4,624,919; 4,670,387; 4,678,747; 4,683,198; 4,683,297; 4,693,974; 4,757,012; 4,770,994; 4,782,019; 4,818,816; 4,835,105; 4,835,264; 4,849,356; 4,851,517; 4,855,128; 4,859,590; 4,865,976; 4,868,104; 4,876,195; 4,900,822; 4,912,093; 4,918,009; 4,925,796; 4,931,389; 4,943,630; 4,957,860; 5,047,335; 5,180,674; 5,288,637; 5,308,460; 5,874,261; and 6,331,418, which are hereby incorporated herein by reference in their entirety for the teaching of carbohydrate synthesis, can be used to provide the disclosed polymers and/or the disclosed saccharide residues.

It has been discovered that polysaccharide sequences can be rapidly and accurately sequenced to identify a signature component of the polysaccharide. The signature component can be used to characterize the polysaccharide sample in ways that were not previously possible. For example, U.S. patent application Ser. No. 09/951,138 provides a method for characterizing samples of polysaccharides, which is incorporated by reference herein for the teaching of polysaccharide sequencing and design. This system can be used for detailed structural analysis (sequencing) of complex sugar-based products in order to design generic versions of these sugars. See Momenta Pharmaceuticals, Inc. (http ://www.momentapharma.com/index.htm).

### E. Definitions

Unless defined otherwise, all technical and scientific terms used herein have the same meanings as commonly understood by one of skill in the art to which the disclosed method and compositions belong.

It must be noted that as used herein and in the appended claims, the singular forms “a,” “an,” and “the” include plural reference unless the context clearly dictates otherwise. Thus, for example, reference to “a polymer” includes a plurality of such polymers, reference to “the polymer” is a reference to one or more polymers and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application data is provided in a number of different formats and that this data represents endpoints and starting points and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Throughout the description and claims of this specification, the word “comprise” and variations of the word, such as “comprising” and “comprises,” means “including but not limited to,” and is not intended to exclude, for example, other additives, components, integers or steps.

As used herein, the term “subject” means any target of administration. The subject can be a vertebrate, for example, a mammal. Thus, the subject can be a human. The term does not denote a particular age or sex. Thus, adult and newborn subjects, whether male or female, are intended to be covered. A patient refers to a subject afflicted with a disease or disorder. The term “patient” includes human and veterinary subjects.

As used herein, the term “biocompatible” refers to materials, or by-products thereof, that are non-toxic and do not elicit a strong immunological reaction against the material. However, the term “biocompatible” does not necessarily exclude materials that elicit an immunogenic response such that the reaction is not adverse.

As used herein, the term “biodegradable” refers to materials which are enzymatically or chemically degraded, or degraded by dissociative processes such as unlinking of an ionically cross-linked material, or dissociation of physically cross-linked structures in vivo into simpler chemical species or species that can be processed by the body through excretory mechanism's.

The term “anti-angiogenic agent” refers to a composition that is capable of reducing the formation or growth of new blood vessels and/or sprouting from existing blood vessels.

As used herein, the terms “implanting” or “implantation” refer to any method of introducing a composition, for example a biocompatible hydrogel, into a subject. Such methods are well known to those skilled in the art and include, but are not limited to, surgical implantation or endoscopic implantation. The term can include both sutured and bound implantation.

By “effective amount” is meant an amount sufficient for performing the desired function or property in a given volume or dimension of tissue for which an effective amount is expressed. As will be pointed out below, the exact amount required will vary from process to process, depending on recognized variables such as the compounds employed and the processing conditions observed. Thus, it is not possible to specify an exact “effective amount.” However, an appropriate effective amount may be determined by one of ordinary skill in the art using only routine experimentation. For example, a therapeutically effective amount of a biocompatible hydrogel disclosed herein can be an amount sufficient to stimulate chondrocyte formation, wherein the chondrocytes are either themselves therapeutic or can be used in a subsequent treatment. For example, a effective amount of a biocompatible hydrogel disclosed herein can be an amount sufficient to stimulate chondrocyte formation, wherein the chondrocytes are either themselves therapeutic or can be used in a subsequent treatment.

By “therapeutically effective” amount is meant an amount that is sufficient to achieve the desired therapeutic result or to have an effect on undesired symptoms, but is generally insufficient to cause adverse side affects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of bioactive or pharmaceutical products.

### F. EXAMPLES

### 1. EXAMPLE 1

### Methods

Preparation of Liposomes: Multi-walled liposomes composed of dioleyl-phosphatidylcholine, cholesterol, cardiolipin, and triolein were prepared as described with some minor modifications (Hunziker, E. B. et al, 2003; Kim, S., et al, 1983). The final liposome preparation contained 60 ng/mL of TGFβ1, 0.4 M Suramin and had a mean diameter of 50 nm. 250 μL of this solution was mixed with 1 mL of Hyaluronic Gel (HA, Sepra Gel) (Genzyme, USA).

Detailed Preparation of Liposomes: The liposomes were prepared as described elsewhere (Hunziker, E. B. and Driesang, I. M., Osteoarthritis Cartilage 11 (5), 320 (2003); Kim, S., Turker, M. S., Chi, E. Y. et al., Biochim Biophys Acta 728 (3), 339 (1983)) with some minor modifications. Briefly, 8.9 μM dioleyl-phosphatidylcholine (DOPC), 8 μM cholesterol (C), 1.5 μM cardiolipin (CL), and 0.1 μM triolein (T), all purchased from Sigma Chemicals (St. Louis, Mo.), were dissolved in 0.5 mL chloroform and then mixed with equal volume of anhydrous ethyl ether in a scintillation vial. 1 mL of 0.15 M aqueous sucrose solution containing ˜200 ng of TGFβ1 (R&D Sciences) and 0.4 M Suramin was then added to the organic phase over 5 seconds under an argon atmosphere with constant agitation. The contents of the scintillation vial was then gently vortexed for 5-10 minutes to create a milky-white water/lipid emulsion. The emulsion was then draw through a 25-gauge needle using a 5 mL syringe few times to size the liposomes and then rapidly introduced into 2.5 mL of 0.2 M sucrose solution placed in a scintillation vial. The contents of the scintillation vial were then transferred to a 250 mL Erlenmeyer flask and the organic phase was evaporated under constant agitation using repeated cycles of vacuum followed by argon flushing until the solution became clear. The liposomes were then pelletized by centrifugation after dilution with 1× PBS (500 g for 5 minutes) and then resuspended in 3 mL PBS.

Preparation of Agarose-PRP gels: 10 mL of autologous blood of each rabbit was used for preparation of Platelet Rich Plasma (PRP) as described (Hunziker E B, et al, 2003). After 3 freeze-taw cycles, 550 μL of PRP was mixed with 550 μL of 4% low melting agarose (30° C., Invitrogen). As a control 2% low melting agarose (30° C., Invitrogen) or HA gel (Genzyme USA) without TGFβ1/Suramin was used.

In vivo Engineering of Cartilage: Skeletally mature female New Zealand white rabbits were used for this study. The IVB was created as described with some minor modifications (Stevens, M. M., et al, 2005; Marini, R. P., et al, 2004). Specifically, the periosteum was exposed by incision of proximal part of the pes ancersinus while keeping the tendon of the semitendinosus muscle untouched. Gels were injected and gelation of agarose based gels was enhanced by cooling with 5° C. sterile 0.9% NaCl. The animals were followed up on day 13 for the HA-gel group and on day 20 for the Agarose gel group.

Tissue Isolation: Specimens were cut in two halves, one half was used for RNA isolation, and the other fixed in 4% paraformaldehyde, dehydrated and embedded in paraffin.

RNA Isolation and Real time PCR (RT-PCR): Immediately after harvest, the tissue was frozen in liquid nitrogen, pulverized, and the resulting powder collected in TriZol reagent. RNA was extracted and RT-PCR was performed in triplicate for Collagen Type II (COL2) and normalized to 28 S rRNA.

Staining protocols: Paraffin sections were deparaffinized, hydrated and stained with thionine for 10 minutes and then coverslipped. The monoclonal anti-COL2 mouse antibody (IIII6B3, Developmental Studies Hybridoma Bank, USA) was used to show the presence of COL2 followed by Hematoxylin counterstaining.

### Results

The results from the studies are tabulated in Table 1. Formation of cartilaginous tissue was observed in 5/7 IVBs that were filled with HA-gel+liposomes. In contrast, IVBs filled with only HA-gel yielded no cartilaginous tissue. However, agarose by itself favored chondrogenesis within the IVB. Also, the cross-sectional area of the cartilage in the agarose group was over twice that in the HA-liposome group.

TABLE 1
Outcomes in IVB sites as a function of biomaterial-gel composition

| | | | Crosssectional |
|---|---|---|---|
| Biomaterial in IVB | # IVB sites(n) | Cartilage (%) | Area (μm²) |
| HA Gel | 5 | 0/5 (0) | — |
| HA Gel + Liposomes | 7 | 5/7 (71) | 9468 ± 2439 |
| Agarose | 4 | 4/4 (100) | 24144 ± 11497 |
| Agarose + PRP | 4 | 0/4 (0) | — |

### Discussion

Tissue sections from In vivo Bioreactor (IVB) filled with Hyaluronic acid (HA)-Gel+liposome were stained with COL2. The IVB space was populated by hypertrophic chondrocytes, similar to what is observed in hyaline cartilage when filled with HA-gel and liposomes. The presence of hyaline cartilage was confirmed both by thionine staining, RT-PCR for COL2 mRNA and by positive immunostaining for COL2. This was absent in the control group (HA gel only). While agarose-PRP did not yield any cartilage, agarose by itself was capable of inducing chondrogenesis within the IVB.

### G. REFERENCES

Brittberg M, Lindahl A, Nilsson A, Ohlsson C, Isaksson O, Peterson L. Treatment of deep cartilage defects in the knee with autologous chondrocyte transplantation. N Engl J Med 1994;331(14):889-95.
Brittberg M, Nilsson A, Lindahl A, Ohlsson C, Peterson L. Rabbit articular cartilage defects treated with autologous cultured chondrocytes. Clin Orthop Relat Res 1996(326):270-83.
Caplan M R, Moore P N, Zhang S, Kamm R D, Lauffenburger D A. Biomacromolecules. 2000;1:627-631.
Cook J L, Williams N, Kreeger J M, Peacock J T, Tomlinson J L. Biocompatibility of three dimensional chondrocyte grafts in large tibial defects of rabbits. Am J Vet Res 2003;64(1):12-20.
D'Ambrosia R D, Lane J, Jackson D, Marshall W, Buckwalter J A, Mankin H, Jackson R. Opposing views in the treatment of OA in the knee; 2001 March; San Francisco; 2001.
Gross A E. Cartilage resurfacing: Filling defects. J Arthroplasty 2003;18(3 Suppl 1):14-7.
Gunther M, Waxman D J, Wagner E, Ogris M. Effects of hypoxia and limited diffusion in tumor cell microenvironment on bystander effect of P450 prodrug therapy. Cancer Gene Ther. 2006 August;13(8):771-9.
Helmlinger G, Netti P A, Lichtenbeld H C, Melder R J, Jain R K. Nat Biotech 1997 August:15(8):778-783.) Holmes T C, de Lacalle S, Su X, Liu G, Rich A, Zhang S. Proc Natl Acad Sci USA. 2000;97:6728-6833.
Holmes T C. Trends Biotechnol. 2002;20:16-21.
Hulst, A. C., Hens, H. J. H., Buitelaar, R. M., and Tramper, J. Determination of the effective diffusion coefficient of oxygen in gel materials in relation to gel concentration. 1989. Biotechnology Techniques. 3(3):1573-6784.
Hunziker E B, Driesang I M. Functional barrier principle for growth-factor-based articular cartilage repair. Osteoarthritis Cartilage 2003;1 1(5):320-7.
Hunziker, E. B. and Driesang, I. M., Osteoarthritis Cartilage 11 (5), 320 (2003)
Jung, R. E., Schmoekel, H. G., Zwahlen, R. et al., Clin Oral Implants Res 16 (6), 676 (2005).
Kim, S., Turker, M. S., Chi, E. Y. et al., Biochim Biophys Acta 728 (3), 339 (1983).
Kisiday J, Jin M, Kurz B, Hung H, Semino C, Zhang S, Grodzinsky A J. Proc Natl Acad Sci U S A. 2002 July 23;99(15):9996-10001
Maes C, Coenegrachts M, Stockmans I, Daci E, Luttun A, Petryk A, Gopalakrishnan R, Moermans K, Smets N, Verafaille C M, Carmeliet P, Bouillon R, Carmeliet G. J Clin Invest 2006, May; 116(5): 1230-42.
Marini D M, Hwang W, Lauffenburger D A, Zhang S, Kamm R D. Nanoletters. 2002;2:295-259.
Marini R P, Stevens M M, Langer R, Shastri V P. Hydraulic elevation of the periosteum: a novel technique for periosteal harvest. J Invest Surg 17 (4), 229 (2004).
McCabe M, Laurent T C. Diffucison of OXYGEN, Nitrogen and water in hyluronate solutions. Biochim Biophys Acta. 1975 July 14;399(1):131-8.
Mollenhauer, J. A. and Erdmann, S., Cell Mol Life Sci 59 (1), 3 (2002).
O'Driscoll S W, Recklies A D, Poole A R. Chondrogenesis in periosteal explants. An organ culture model for in vitro study. J Bone Joint Surg Am 1994;76(7):1042-51.
O'Driscoll S W. Articular cartilage regeneration using periosteum. Clin Orthop Relat Res 1999(367 Suppl):S186-203.
O'Driscoll S W. Re: ‘neochondrogenesis in repair of full-thickness articular cartilage defects using free autogenous periosteal grafts in the rabbit. A follow-up in six months’ by carranza-bencano et al. Osteoarthritis Cartilage 2001;9(2):187.
Schaefer, D., Martin, I., Jundt, G. et al., Arthritis Rheum 46 (9), 2524 (2002).
Solchaga, L. A., Goldberg, V. M., and Caplan, A. I., Clin Orthop Relat Res (391 Suppl), S161 (2001).
Stevens M M, Marini R P, Schaefer D, Aronson J, Langer R, Shastri V P. In vivo engineering of organs: The bone bioreactor. Proc Natl Acad Sci U S A 2005;102(32):11450-5.
Stevens, M. M., Marini, R. P., Schaefer, D. et al., Proc Natl Acad Sci U S A 102 (32), 11450 (2005).
Zhang S, Holmes T C, DiPersio C M, Hynes R 0, Su X, Rich A. Biomaterials. 1995;16:1385-1393.
Zhang S, Holmes T, Lockshin C, Rich A. Proc Natl Acad Sci USA. 1993;90:3334-3338.

What is claimed is:

1. A method for promoting generation of cartilage tissue, comprising administering in or adjacent to periosteum tissue of a subject a therapeutically effective amount of a biocompatible hydrogel comprising at least one polymer, wherein exogenous cells are substantially absent, wherein non-carbohydrate anti-angiogenic agents and/or growth factors are substantially absent.

2. The method of claim 1, further comprising administering an angiogenic agent in or adjacent to periosteum tissue or to the biocompatible hydrogel, wherein the method promotes generation of bone tissue.

3. The method of claim 1, wherein the polymer comprises one or more saccharide residues having the structure: 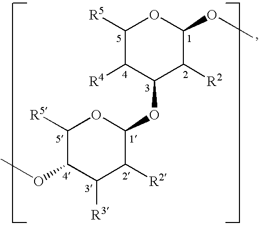
wherein R², R²′, and R³′, are, independently, hydrogen, hydroxyl, alkoxyl, alkylether, amine, or amide;
wherein R⁴ is hydrogen, hydroxyl, alkoxyl, alkylether, amine, or amide; and
wherein R⁵ and R⁵′ are, independently, hydrogen, hydroxyl, alkoxyl, alkyl, hydroxymethylene, alkylether, amine, or amide.

4. The method of claim 3, wherein the polymer comprises one or more saccharide residues having the structure:

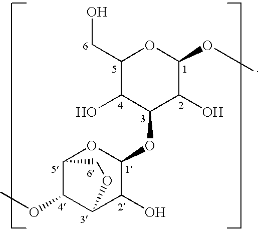

5. The method of claim 1, wherein the biocompatible hydrogel comprises a diffusion coefficient of oxygen less than 1.5×10⁻⁹ m²s⁻¹.

6. The method of claim 1, wherein the biocompatible hydrogel further comprises at least one biocompatible polymer selected from agarose, hyaluronic acid, heparin, a heparin fragment, and mixtures thereof, glycosaminoglycans, glycosylated proteins (proteoglycans), glycosylated non degradable and degradable synthetic polymers, polymers with sugar residues, and self-assemble peptides.

7. The method of claim 1, wherein the biocompatible hydrogel further comprises at least one pharmaceutically active agent.

8. The method of claim 1, wherein the biocompatible hydrogel has a modulus of at least 0.3 megapascals.

9. The method of claim 8, wherein sodium alginate is used to increase modulus of the biocompatible hydrogel.

10. The method of claim 1, wherein the biocompatible hydrogel induces the local production of cytokines, wherein at least one of the cytokines stimulates chondrogenic differentiation of the periosteal cells.

11. The method of claim 1, further comprising the step of creating an artificial space or environment in an organ or cavity of the subject prior to the administration step.

12. The method of claim 11, further comprising the step of harvesting pluripotent or multipotent progenitor cells, chondrocytes, or cartilage from the artificial space or environment after the administration step.

13. The method of claim 12, further comprising the step of re-introducing the harvested progenitor cells, chondrocytes, or cartilage into the subject.

14. A method for promoting generation of cartilage tissue, comprising administering in or adjacent to periosteum tissue of a subject a therapeutically effective amount of a biocompatible hydrogel consisting essentially of agarose and water.

15. The method of claim 14, further comprising administering an angiogenic agent in or adjacent to periosteum tissue or to the biocompatible hydrogel, wherein the method promotes generation of bone tissue.

16. The method of claim 14, further comprising the step of creating an artificial space or environment in an organ or cavity of the subject prior to the administration step.

17. The method of claim 16, further comprising the step of harvesting pluripotent or multipotent progenitor cells, chondrocytes, or cartilage from the artificial space or environment after the administration step.

18. The method of claim 17, further comprising the step of re-introducing the harvested progenitor cells, chondrocytes, or cartilage into the subject.

19. A method for promoting generation of cartilage tissue, comprising the steps of:
(a) creating an artificial space or environment in an organ or cavity of a subject;
(b) administering in or adjacent to periosteum tissue of the subject a therapeutically effective amount of a biocompatible hydrogel, thereby providing a hypoxic environment in the space; and
(c) optionally, harvesting pluripotent or multipotent progenitor cells, chondrocytes, or cartilage from the artificial space or environment after the administration step.

20. A kit for promoting generation of tissue in vivo, comprising a biocompatible hydrogel and two or more of:
(a) a tissue retractor for generating an artificial space at a site in a subject;
(b) an agent to partially degrade connective tissue at the site, thereby freeing cells to promote formation of the space and/or promote migration of cells into the space;
(c) means for warming the hydrogel to a melting temperature; and
(d) means for delivery of the melted gel to the site.

21. The kit of claim 20, wherein the biocompatible hydrogel comprises at least one polymer, wherein exogenous cells are substantially absent, and wherein non-carbohydrate anti-angiogenic agents and/or growth factors are substantially absent.

22. The kit of claim 20, wherein the biocompatible hydrogel consists essentially of agarose and water.

23. The kit of claim 20, comprising;
(a) a biocompatible hydrogel;
(b) a tissue retractor for generating an artificial space at a site in a subject; and
(c) an agent to partially degrade connective tissue at the site, thereby freeing cells to promote formation of the space and/or promote migration of cells into the space.

24. The kit of claim 20, comprising;
(a) a biocompatible hydrogel;
(b) means for warming the hydrogel to a melting temperature; and
(c) means for delivery of the melted gel to the site.

25. The kit of claim 20, wherein the biocompatible hydrogel further comprises at least one pharmaceutically active agent.
